(19) United States
(12) Patent Application Publication
(10) Pub. No.: US 2010/0112052 A1
(43) Pub. Date: May 6, 2010

(54) OSMOTIC TABLET WITH A COMPRESSED OUTER COATING

(76) Inventors: Vincent Chen, Dayton, NJ (US); Shun-Por Li, Lansdale, PA (US); Robert Shen, North Wales, PA (US); Der-Yang Lee, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/607,248

(22) Filed: Oct. 28, 2009

Related U.S. Application Data
(60) Provisional application No. 61/110,022, filed on Oct. 31, 2008.

(51) Int. Cl. A61K 9/22; A61K 31/135; A61K 31/495

(52) U.S. Cl. 424/468; 427/2.14; 514/653; 514/255.04

(57) ABSTRACT

The present invention features a method of manufacturing an osmotic tablet including the steps of (i) compressing a tablet core including a first pharmaceutically active agent and a hydrophilic polymer; (ii) applying an osmotic coating to the outer surface of the tablet core to form a coated tablet, wherein the osmotic coating includes at least one opening exposing the tablet core; and (iii) compressing an immediate release coating onto the surface of the coated tablet, wherein the release coating includes a second pharmaceutically active agent.

### CROSS REFERENCE TO RELATED APPLICATION

This application claims priority of the benefits of the filing of U.S. Provisional Application Ser. No. 61/110,022, filed Oct. 31, 2008. The complete disclosure of the aforementioned related U.S. patent application is hereby incorporated herein by reference for all purposes.

### BACKGROUND OF THE INVENTION

The separation of pharmaceutically active agents from one another within tablets is often necessary to prevent degradation. Osmotic tablets have been traditionally used to deliver pharmaceutically active agents in a sustained release manner, such as a zero order manner. When a second pharmaceutically active agent or second portion of the same pharmaceutically active agent is incorporated into the tablet, it is often desirable to have this active be delivered in an immediate release manner. In order to achieve this result, the pharmaceutically active agent must often be applied on the outside of the osmotic tablet, e.g., using spray coating, as shown in such references as U.S. Pat. No. 6,919,373. However, this process can present several issues. Large dose pharmaceutically active agents can be problematic in spray coating since they often require long spray times due to the large quantity of solution to accommodate the large concentration. Additionally, certain pharmaceutically active agent may not be compatible with certain solution solvents, such as water, which may lead to degradation of the agent. Finally, since pharmaceutically active agent must often recrystallize out of solution upon spraying, there must be assurance that the crystal structure of the pharmaceutically active agent does not substantially change. Cetirizine is especially susceptible to degradation in aqueous solutions and in combination with certain compounds such as sympathomimetic amines. In these combinations, cetirizine can degrade in terms of formation of undesirable cetirizine esters and cetirizine oxidative degradants.

The present invention relates to a novel method of manufacturing an osmotic tablet including a tablet core with both an osmotic coating and a compressed immediate release coating.

### SUMMARY OF THE INVENTION

The present invention features a method of manufacturing an osmotic tablet including the steps of (i) compressing a tablet core including a first pharmaceutically active agent and a hydrophilic polymer; (ii) applying an osmotic coating to the outer surface of the tablet core to form a coated tablet, wherein the osmotic coating includes at least one opening exposing the tablet core; and (iii) compressing an immediate release coating onto the surface of the coated tablet, wherein the release coating includes a second pharmaceutically active agent.

The present invention also features an osmotic tablet manufactured according to such method and a method of administering a first pharmaceutically active agent and a second pharmaceutically active agent, the method including ingesting such osmotic tablet.

Other features and advantages of the present invention will be apparent from the detailed description of the invention and from the claims.

### DETAILED DESCRIPTION OF THE INVENTION

It is believed that one skilled in the art can, based upon the description herein, utilize the present invention to its fullest extent. The following specific embodiments can be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified.

The tablets of the present invention contain one or more pharmaceutically active agents that are released therefrom upon contact of the tablet with a liquid medium, for example a dissolution medium such as gastrointestinal fluids.

“Water soluble,” as used herein in connection with non-polymeric materials, shall mean from sparingly soluble to very soluble, i.e., not more than 100 parts water required to dissolve 1 part of the non-polymeric, water soluble solute. See Remington, The Science and Practice of Pharmacy, pp 208-209 (2000). “Water soluble,” as used herein in connection with polymeric materials, shall mean that the polymer swells in water and can be dispersed at the molecular level or dissolved in water.

As used herein, the term “modified release” shall apply to tablets, matrices, particles, coatings, portions thereof, or compositions that alter the release of an pharmaceutically active agent in any manner. Types of modified release include controlled, prolonged, sustained, extended, delayed, pulsatile, repeat action, and the like. Suitable mechanisms for achieving these types of modified release include diffusion, erosion, surface area control via geometry and/or impermeable barriers, or other mechanisms known in the art.

### Manufacture of Tablet Core

In one embodiment of the invention, the first pharmaceutically active agent and the hydrophilic polymer are mixed with a powder containing a pharmaceutically-acceptable carrier, which is also defined herein as the tablet matrix. In one embodiment, the powder has an average particle size of about 50 microns to about 500 microns, such as between 50 microns and 300 microns. Particles in this size range are particularly useful for direct compression processes.

In embodiment, the components of powder are blended together, for example as dry powders, and fed into the die cavity of an apparatus that applies pressure to form a tablet core. Any suitable compacting apparatus may be used, including, but not limited to, conventional unitary or rotary tablet press. In one embodiment, the tablet core may be formed by compaction using a rotary tablet press (e.g., such as those commercially available from Fette America Inc., Rockaway, N.J., or Manesty Machines LTD, Liverpool, UK). In general, a metered volume of powder is filled into a die cavity (where the powder is either gravity fed or mechanically fed from a feeder) of the rotary tablet press, and the cavity rotates as part of a “die table” from the filling position to a compaction position. At the compaction position, the powder is compacted between an upper and a lower punch, then the resulting tablet core is pushed from the die cavity by the lower punch and then guided to an injection chute by a stationary “take-off” bar.

In another embodiment, the tablet may be prepared by the compression methods and apparatus described in United States Patent Application Publication No. 20040156902. Specifically, the tablet core may be made using a rotary compression module including a fill zone, insertion zone, compression zone, ejection zone, and purge zone in a single apparatus having a double row die construction. The dies of the compression module may then be filled using the assistance of a vacuum, with filters located in or near each die. The purge zone of the compression module includes an optional powder recovery system to recover excess powder from the filters and return the powder to the dies.

In another embodiment, the tablet matrix may be prepared by a wet-granulation method, in which the excipients and a solution or dispersion of a wet binder (e.g., an aqueous cooked starch paste or solution of polyvinyl pyrrolidone) are mixed and granulated. Suitable apparatus for wet granulation include, but are not limited to, low shear mixers (e.g., planetary mixers), high shear mixers, and fluid beds (including rotary fluid beds). The resulting granulated material may then be dried, and optionally dry-blended with further ingredients (e.g., excipients such as lubricants, colorants, and the like). The final dry blend is then suitable for compression by the methods described in the previous paragraph.

In one embodiment, the tablet core is prepared by the compression methods and apparatus described in issued U.S. Pat. No. 6,767,200. Specifically, the tablet core is made using a rotary compression module including a fill zone, compression zone, and ejection zone in a single apparatus having a double row die construction as shown in FIG. 6 therein. The dies of the compression module are preferably filled using the assistance of a vacuum, with filters located in or near each die.

In one embodiment of the invention, the tablet core may be a directly compressed tablet core made from a powder that is substantially free of water-soluble polymeric binders and hydrated polymers. As used herein, what is meant by “substantially free” is less than 5 percent, such as less than 1 percent, such as less than 0.1 percent, such as completely free (e.g., 0 percent). This composition is advantageous for minimizing processing and material costs and providing for optimal physical and chemical stability of the tablet core. In one embodiment, the density of the tablet core is greater than about 0.9 g/cc.

The tablet core may have one of a variety of different shapes. For example, the tablet core may be shaped as a polyhedron, such as a cube, pyramid, prism, or the like; or may have the geometry of a space figure with some non-flat faces, such as a cone, truncated cone, cylinder, sphere, torus, or the like. In certain embodiments, a tablet core has one or more major faces. For example, the tablet core surface typically has opposing upper and lower faces formed by contact with the upper and lower punch faces in the compression machine. In such embodiments the tablet core surface typically further includes a “belly-band” located between the upper and lower faces, and formed by contact with the die walls in the compression machine.

### Hydrophilic Polymer and Osmogen

As discussed above, the tablet core contains one or more hydrophilic polymers. Suitable hydrophilic polymers include, but are not limited to, water swellable cellulose derivatives, polyalkylene glycols, thermoplastic polyalkylene oxides, acrylic polymers, hydrocolloids, clays, gelling starches, swelling cross-linked polymers, and mixtures thereof. Examples of suitable water swellable cellulose derivatives include, but are not limited to, sodium carboxymethylcellulose, cross-linked hydroxypropylcellulose, hydroxypropyl cellulose (HPC), hydroxypropylmethylcellulose (HPMC), hydroxyisopropylcellulose, hydroxybutylcellulose, hydroxyphenylcellulose, hydroxyethylcellulose (HEC), hydroxypentylcellulose, hydroxypropylethylcellulose, hydroxypropylbutylcellulose, and hydroxypropylethylcellulose, and mixtures thereof. Examples of suitable polyalkylene glycols include, but are not limited to, polyethylene glycol. Examples of suitable thermoplastic polyalkylene oxides include, but are not limited to, poly(ethylene oxide). Examples of suitable acrylic polymers include, but are not limited to, potassium methacrylatedivinylbenzene copolymer, polymethylmethacrylate, high-molecular weight cross-linked acrylic acid homopolymers and copolymers such as those commercially available from Noveon Chemicals under the tradename CARBOPOL™ (e.g., having a viscosity of greater than 50,000 centipoise when tested using a Brookfield RVT Viscometer at 25° C., using spindle # 7, when dispersed in a basic solution). Examples of suitable hydrocolloids include, but are not limited to, alginates, agar, guar gum, locust bean gum, kappa carrageenan, iota carrageenan, tara, gum arabic, tragacanth, pectin, xanthan gum, gellan gum, maltodextrin, galactomannan, pusstulan, laminarin, scleroglucan, gum arabic, inulin, pectin, gelatin, whelan, rhamsan, zooglan, methylan, chitin, cyclodextrin, chitosan, and mixtures thereof. Examples of suitable clays include, but are not limited to, smectites such as bentonite, kaolin, and laponite; magnesium trisilicate; magnesium aluminum silicate; and mixtures thereof. Examples of suitable gelling starches include, but are not limited to, acid hydrolyzed starches, swelling starches such as sodium starch glycolate and derivatives thereof, and mixtures thereof. Examples of suitable swelling cross-linked polymers include, but are not limited to, cross-linked polyvinyl pyrrolidone, cross-linked agar, and cross-linked carboxymethylcellulose sodium, and mixtures thereof.

In one embodiment, an osmogen is incorporated into the tablet core in order to draw water into the tablet upon contact with fluids, such as gastrointestinal fluids. An osmogen as used herein is a water soluble component which preferentially draws water into the tablet core for the purposes of distributing the water throughout the core, so that the active ingredient contained in the core may be released. In one embodiment the osmogen is a salt such as but not limited to sodium chloride, potassium chloride, sodium citrate, or potassium citrate.

### Pharmaceutically-Acceptable Carrier

As discussed above, the tablet core is manufactured by compressing a powder containing a pharmaceutically-acceptable carrier. The carrier may contain one or more suitable excipients for the formulation of tablets. Examples of suitable excipients include, but are not limited to, fillers, adsorbents, binders, disintegrants, lubricants, glidants, release-modifying excipients, superdisintegrants, antioxidants, and mixtures thereof.

Suitable fillers include, but are not limited to, water-soluble compressible carbohydrates such as sugars (e.g., dextrose, sucrose, maltose, and lactose), starches (e.g., corn starch), sugar-alcohols (e.g., mannitol, sorbitol, maltitol, erythritol, and xylitol), starch hydrolysates (e.g., dextrins, and maltodextrins), and water insoluble plastically deforming materials (e.g., microcrystalline cellulose or other cellulosic derivatives), and mixtures thereof.

Suitable adsorbents (e.g., to adsorb the liquid drug composition) include, but are not limited to, water-insoluble adsorbents such as dicalcium phosphate, tricalcium phosphate, silicified microcrystalline cellulose (e.g., such as distributed under the PROSOLV brand (PenWest Pharmaceuticals, Patterson, N.Y.)), magnesium aluminometasilicate (e.g., such as distributed under the NEUSILIN™ brand (Fuji Chemical Industries (USA) Inc., Robbinsville, N.J.), clays, silicas, bentonite, zeolites, magnesium silicates, hydrotalcite, veegum, and mixtures thereof.

Suitable binders include, but are not limited to, dry binders such as polyvinyl pyrrolidone and hydroxypropylmethylcellulose; wet binders such as water-soluble polymers, including hydrocolloids such as acacia, alginates, agar, guar gum, locust bean, carrageenan, carboxymethylcellulose, tara, gum arabic, tragacanth, pectin, xanthan, gellan, gelatin, maltodextrin, galactomannan, pusstulan, laminarin, scleroglucan, inulin, whelan, rhamsan, zooglan, methylan, chitin, cyclodextrin, chitosan, polyvinyl pyrrolidone, cellulosics, sucrose, and starches; and mixtures thereof.

Suitable disintegrants include, but are not limited to, sodium starch glycolate, cross-linked polyvinylpyrrolidone, cross-linked carboxymethylcellulose, starches, microcrystalline cellulose, and mixtures thereof.

Suitable lubricants include, but are not limited to, long chain fatty acids and their salts, such as magnesium stearate and stearic acid, talc, glycerides waxes, and mixtures thereof.

Suitable glidants include, but are not limited to, colloidal silicon dioxide.

Suitable release-modifying excipients include, but are not limited to, insoluble edible materials, pH-dependent polymers, and mixtures thereof.

Suitable insoluble edible materials for use as release-modifying excipients include, but are not limited to, water-insoluble polymers and low-melting hydrophobic materials, copolymers thereof, and mixtures thereof. Examples of suitable water-insoluble polymers include, but are not limited to, ethylcellulose, polyvinyl alcohols, polyvinyl acetate, polycaprolactones, cellulose acetate and its derivatives, acrylates, methacrylates, acrylic acid copolymers, copolymers thereof, and mixtures thereof. Suitable low-melting hydrophobic materials include, but are not limited to, fats, fatty acid esters, phospholipids, waxes, and mixtures thereof. Examples of suitable fats include, but are not limited to, hydrogenated vegetable oils such as for example cocoa butter, hydrogenated palm kernel oil, hydrogenated cottonseed oil, hydrogenated sunflower oil, and hydrogenated soybean oil, free fatty acids and their salts, and mixtures thereof. Examples of suitable fatty acid esters include, but are not limited to, sucrose fatty acid esters, mono-, di-, and tri-glycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, stearoyl macrogol-32 glycerides, and mixtures thereof. Examples of suitable phospholipids include phosphotidyl choline, phosphotidyl serene, phosphotidyl enositol, phosphotidic acid, and mixtures thereof. Examples of suitable waxes include, but are not limited to, carnauba wax, spermaceti wax, beeswax, candelilla wax, shellac wax, microcrystalline wax, and paraffin wax; fat-containing mixtures such as chocolate, and mixtures thereof.

Examples of superdisintegrants include, but are not limited to, croscarmellose sodium, sodium starch glycolate and cross-linked povidone (crospovidone). In one embodiment the tablet core contains up to about 5 percent by weight of such superdisintegrant.

Examples of antioxidants include, but are not limited to, tocopherols, ascorbic acid, sodium pyrosulfite, butylhydroxytoluene, butylated hydroxyanisole, edetic acid, and edetate salts, and mixtures thereof. Examples of preservatives include, but are not limited to, citric acid, tartaric acid, lactic acid, malic acid, acetic acid, benzoic acid, and sorbic acid, and mixtures thereof.

### Application of Osmotic Coating

The osmotic tablets of the present invention include an osmotic coating. An osmotic coating is one that is semipermeable thereby allows water to be drawn into the tablet core, e.g., for the purposes of releasing the active ingredient such as through a pre-made hole in the coating or through coating itself it is semipermeable membrane. The osmotic coating, thus, does not fully dissolve upon contact with water In one embodiment, the osmotic coating contains a water soluble component such as a water solible film former which aids in facilitating a further influx of water upon contact with water. In the current invention the osmotic coating is applied via spray coating. Suitable spray coating techniques include spray coating via a coating pan or fluid bed process such as Wurster coating or top spray fluid bed coating as described in the text, “The Theory and Practice of Industrial Pharmacy”, Lachman, Leon et. al, 3rd ed. The osmotic coating may be applied using a solution prepared with water, organic solvents, or mixtures thereof. Suitable organic solvents include but are not limited to acetone, isopropanol, methylene chloride, hexane, methanol, ethanol, and mixtures thereof. In one embodiment the polymer(s) are dissolved in the coating solution. In one embodiment, the polymer(s) are dispersed, as is the case when applying water insoluble polymers via a dispersion or as is the case when using ethylcellulose dispersions.

In one embodiment in which the osmotic coating functions as a semipermeable membrane (e.g., allowing water or solvent to pass into the core, but being impermeable to dissolved pharmaceutically active agent, thereby preventing the passage of pharmaceutically active agent therethrough) the film former is selected from water insoluble polymers, pH-dependent polymers, water soluble polymers, and combinations thereof. In one embodiment, the osmotic coating includes a water insoluble polymer and a pore forming material. Examples of suitable water-insoluble polymers include ethylcellulose, polyvinyl alcohols, polyvinyl acetate, polycaprolactones, cellulose acetate and its derivatives, acrylates, methacrylates, acrylic acid copolymers, and combinations thereof. In one embodiment, the water insoluble polymer is cellulose acetate. In one embodiment, the osmotic coating includes from about 10 to about 100 weight percent of a water insoluble film former.

In one embodiment of the osmotic coating, the water insoluble polymer is combined with a water soluble film former in order to create pores in the resulting semi-permeable membrane. Examples of suitable film formers include, but are not limited to: water soluble vinyl polymers such as polyvinylalcohol (PVA); water soluble polycarbohydrates such as hydroxypropyl starch, hydroxyethyl starch, pullulan, methylethyl starch, carboxymethyl starch, pre-gelatinized starches, and film-forming modified starches; water swellable cellulose derivatives such as hydroxypropyl cellulose (HPC), hydroxypropylmethyl cellulose (HPMC), methyl cellulose (MC), hydroxyethylmethylcellulose (HEMC), hydroxybutylmethylcellulose (HBMC), hydroxyethylethylcellulose (HEEC), and hydroxyethylhydroxypropylmethyl cellulose (HEMPMC); water soluble copolymers such as methacrylic acid and methacrylate ester copolymers, polyvinyl alcohol and polyethylene glycol copolymers, polyethylene oxide and polyvinylpyrrolidone copolymers; and mixtures thereof.

In one embodiment, a pH dependent polymer is incorporated into the osmotic coating. In one embodiment, the pH dependent polymer is used at a level of from about 10 to about 50 percent by weight of the osmotic coating. Suitable film-forming pH-dependent polymers include, but are not limited to, enteric cellulose derivatives, such as for example hydroxypropyl methylcellulose phthalate, hydroxypropyl methylcellulose acetate succinate, and cellulose acetate phthalate; natural resins such as shellac and zein; enteric acetate derivatives such as polyvinylacetate phthalate, cellulose acetate phthalate, and acetaldehyde dimethylcellulose acetate; and enteric acrylate derivatives such as for example polymethacrylate-based polymers such as poly(methacrylic acid, methyl methacrylate) 1:2 (commercially available from Rohm Pharma GmbH under the tradename EUDRAGIT S™), and poly(methacrylic acid, methyl methacrylate) 1:1 (commercially available from Rohm Pharma GmbH under the tradename EUDRAGIT L™); and combinations thereof.

In one embodiment, the osmotic coating has an average thickness of at least 5 microns, such as from about 10 microns to about 200 microns, e.g. from about 20 microns to about 150 microns, e.g. from about 30 to about 150 microns. In one embodiment, the osmotic coating is free of porosity (e.g., wherein the pore volume is in a pore diameter range of less than 0.01 g/cc). In one embodiment, the average pore diameter of the osmotic coating is less than about 0.2 microns (e.g., less than about 0.15 microns).

In one embodiment, the osmotic coating is substantially free of an pharmaceutically active agent. In one embodiment the osmotic coating includes an pharmaceutically active agent which is different than the pharmaceutically active agent included in the immediate release coating.

In one embodiment, the osmotic coating includes a plasticizer. In one embodiment the plasticizer must be of sufficient quantity to withstand the compression force of the immediate release coating. Suitable plasticizers include, but are not limited to: polyethylene glycol; propylene glycol; glycerin; sorbitol; triethyl citrate; tributyl citrate; dibutyl sebecate; vegetable oils such as castor oil, grape oil, olive oil, and sesame oil; surfactants such as polysorbates, sodium lauryl sulfates, and dioctyl-sodium sulfosuccinates; mono acetate of glycerol; diacetate of glycerol; triacetate of glycerol; natural gums; triacetin; acetyltributyl citrate; diethyloxalate; diethylmalate; diethyl fumarate; diethylmalonate; dioctylphthalate; dibutylsuccinate; glycerol tributyrate; hydrogenated castor oil; fatty acids such as lauric acid; glycerides such as mono-, di-, and/or triglycerides, which may be substituted with the same or different fatty acids groups such as, for example, stearic, palmitic, and oleic and the like; and mixtures thereof. In one embodiment, the plasticizer is triethyl citrate.

In one embodiment, at least about 50 percent of the cross-sectional area of the osmotic coating used in tablets of this invention is striated, such as at least about 80% of the cross-sectional area of the osmotic coating portion is striated. As used herein, “striated” means non-homogeneous with respect to appearance and with respect to the internal structure of the coating portion when viewed under any magnification and lighting conditions, at which point striations or layers can be viewed. Compressed portions of a pharmaceutical oral dosage forms do not display striated areas, wherein spray coated portions display striations. For example a cross-section of the osmotic coating portion is striated, and non-uniform with respect to refractive properties when observed utilizing a light microscope or a scanning electron microscope at a magnification of about 50 to about 400 times.

The characteristic striations are indicative of the spray-coating process consisting of multiple repetitions of the steps consisting of: (a) application via spraying of coating solution; followed by (b) warm air drying, to a tumbling bed of tablets in a revolving coating pan such that numerous layers of coating material are built up as each application of coating material dries to form a layer. In one embodiment, the thickness of an individual striated layer is the range of about 10 microns to about 15 microns.

In one embodiment, the opening(s) in the osmotic coating may be made with a laser or through mechanical means such as those described in U.S. Pat. No. 7,404,708. In certain embodiments, the osmotic coating is semipermeable (e.g., containing a plurality of small opening) and does not require the addition of an additional opening via laser or other means. In one such embodiment, the semi-permeable membrane of the osmotic coating also allows for the release of the active ingredient in the tablet core through the membrane in a zero-order or first-order release manner.

In certain embodiments the opening(s) in the osmotic coating is not produced through laser drilling, but rather is created through other mechanical means, such as when using the punch assembly in U.S. Pat. No. 7,404,708. Other means for producing the opening include molding of the osmotic coating wherein the mold has a pre-formed opening. In these instances, the osmotic coating may be performed using such equipment as disclosed and described more fully in U.S. Pat. No. 6,767,200, U.S. Patent Applications 2003/008367, 2003/0086973 A1, and 2005/0074514. Still other means for creating the opening may include but are not limited to mechanical drilling, etching, and vacuum removal of the coating portion intended for the opening.

### Compression of Immediate Release Coating

In one embodiment, the immediate release coating has an average thickness of at least 50 microns, such as from about 50 microns to about 2500 microns; e.g., from about 250 microns to about 1000 microns.

In embodiment, the immediate release coating is typically compressed at a density of more than about 0.9 g/cc., as measured by the weight and volume of that specific layer.

In one embodiment, the immediate release coating contains a first portion and a second portion, wherein at least one of the portions contains the second pharmaceutically active agent. In one embodiment, the portions contact each other at a center axis of the tablet. In one embodiment, the first portion includes the first pharmaceutically active agent and the second portion includes the second pharmaceutically active agent.

In one embodiment, the first portion contains the first pharmaceutically active agent and the second portion contains the second pharmaceutically active agent. In one embodiment, one of the portions contains a third pharmaceutically active agent. In one embodiment one of the portions contains a second immediate release portion of the same pharmaceutically active agent as that contained in the tablet core. In one embodiment the tablet core includes a decongestant, the osmotic coating does not include an pharmaceutically active agent, a first outer compressed portion includes an antihistamine and a second outer compressed portion includes a decongestant. In one embodiment the tablet core includes pseudoephedrine, and the outer compressed portion includes cetirizine.

In one embodiment, the outer coating portion is prepared as a dry blend of materials prior to addition to the coated tablet core. In another embodiment the outer coating portion is included of a dried granulation including the pharmaceutically active agent. In one embodiment, the immediate release layers are compressed in two steps, even if the top and bottom layers include the same formulation. In one embodiment, the first compressed layer may be compressed at forces from about 0.5 kiloNewtons to about 10 kiloNewtons, such as from about 0.5 kiloNewtons to about 2 kiloNewtons. In one embodiment, the second outer compressed layer and tablet may be compressed at forces from about 5 kiloNewtons to about 20 kiloNewtons, such as from about 10 kiloNewtons to about 20 kiloNewtons.

In one embodiment, a suitable flavor or aroma agent may be added to the outer coating. Examples of suitable flavor and aroma agents include, but are not limited to, essential oils including distillations, solvent extractions, or cold expressions of chopped flowers, leaves, peel or pulped whole fruit containing mixtures of alcohols, esters, aldehydes and lactones; essences including either diluted solutions of essential oils, or mixtures of synthetic chemicals blended to match the natural flavor of the fruit (e.g., strawberry, raspberry, and black currant); artificial and natural flavors of brews and liquors (e.g., cognac, whisky, rum, gin, sherry, port, and wine); tobacco, coffee, tea, cocoa, and mint; fruit juices including expelled juice from washed, scrubbed fruits such as lemon, orange, and lime; mint; ginger; cinnamon; cacoe/cocoa; vanilla; liquorice; menthol; eucalyptus; aniseeds nuts (e.g., peanuts, coconuts, hazelnuts, chestnuts, walnuts, and colanuts); almonds; raisins; and powder, flour, or vegetable material parts including tobacco plant parts (e.g., the genus Nicotiana in amounts not contributing significantly to a level of therapeutic nicotine), and mixtures thereof.

### Porosity

In one embodiment, the immediate release coating has a porosity (described as an pore volume, which is expressed as cc or cc/gram when normalized for weight) of at least 0.02 cc/g, such as from about 0.02 to about 0.06 cc/g. In one embodiment, the immediate release coating has an average pore diameter of at least about 0.2 microns, such as from about 0.2 to about 5 microns, such as from about 0.2 microns to about 3 microns, such as from about 0.45 microns to about 3 microns. In one embodiment, the osmotic coating is substantially free of pores. In one embodiment, the osmotic coating has a pore volume of less than 0.02 cc/g. In one embodiment, the immediate release compressed coating has a average pore volume that is at least 20 percent greater than the osmotic coating layer. In one embodiment the immediate release compressed coating has an average pore diameter that is at least 20 percent greater than that of the osmotic coating layer. In one embodiment, the immediate release compressed coating has an average percent porosity that is at least 20 percent greater than that of the osmotic coating layer.

Pore volume (expressed in cc or cc/g), pore diameter (expressed in microns) and pore density may be determined using a Quantachrome Instruments PoreMaster 60 mercury intrusion porosimeter and associated computer software program known as “Porowin.” The procedure is documented in the Quantachrome Instruments PoreMaster Operation Manual. The PoreMaster determines both pore volume and pore diameter of a solid or powder by forced intrusion of a non-wetting liquid (mercury), which involves evacuation of the sample in a sample cell (penetrometer), filling the cell with mercury to surround the sample with mercury, applying pressure to the sample cell by: (i) compressed air (up to 50 psi maximum) and (ii) a hydraulic (oil) pressure generator (up to 60000 psi maximum). Intruded volume is measured by a change in the capacitance as mercury moves from outside the sample into its pores under applied pressure. The corresponding pore size diameter (d) at which the intrusion takes place is calculated directly from the so-called “Washburn Equation”: d=−(4γ(cosθ)/P) where γ is the surface tension of liquid mercury, θ is the contact angle between mercury and the sample surface, and P is the applied pressure.

In one embodiment, the porosity of the immediate release layer is measured as follows. The equipment used for pore volume measurements include (1) Quantachrome Instruments PoreMaster 60; (2) Analytical Balance capable of weighing to 0.0001 g.; and (3) Desiccator. The reagents used for measurements include (1) High purity nitrogen; (2) Triply distilled mercury; (2) High pressure fluid (Dila AX, available from Shell Chemical Co.); (3) Liquid nitrogen (for Hg vapor cold trap); (4) Isopropanol or methanol for cleaning sample cells; and (5) Liquid detergent for cell cleaning.

The samples remain in sealed packages or as received in the dessicator until analysis. The vacuum pump is switched on, the mercury vapor cold trap is filled with liquid nitrogen, the compressed gas supply is regulated at 55 psi., and the instrument is turned on and allowed a warm up time of at least 30 minutes. The empty penetrometer cell is assembled as described in the instrument manual and its weight is recorded. The cell is installed in the low pressure station and “evacuation and fill only” is selected from the analysis menu, and the following settings are employed: (1) Fine Evacuation time: 1 min.; (2) Fine Evacuation rate: 10; and (3) Coarse Evacuation time: 5 min.

The cell (filled with mercury) is then removed and weighed. The cell is then emptied into the mercury reservoir, and two tablets from each sample are placed in the cell and the cell is reassembled. The weight of the cell and sample are then recorded. The cell is then installed in the low-pressure station, the low-pressure option is selected from the menu, and the following parameters are set: (1) Mode: Low pressure; (2) Fine evacuation rate: 10 Hg; (3) Fine evacuation until: 200 Hg; (4) Coarse evacuation time: 10 min.; (5) Fill pressure: Contact+0.1; (6) Maximum pressure: 50; (7) Direction: Intrusion And Extrusion; (8) Repeat: 0; (9) Mercury contact angle: 140; and (10) Mercury surface tension: 480

Data acquisition is then begun. The pressure vs. cumulative volume-intruded plot is displayed on the screen. After low-pressure analysis is complete, the cell is removed from the low-pressure station and reweighed. The space above the mercury is filled with hydraulic oil, and the cell is assembled and installed in the high-pressure cavity. The following settings are used: (1) Mode: Fixed rate; (2) Motor speed: 5; (3) Start pressure: 20; (4) End pressure: 60,000; (5) Direction: Intrusion and extrusion; (6) Repeat: 0; (7) Oil fill length: 5; (8) Mercury contact angle: 140; and (9) Mercury surface tension: 480

Data acquisition is then begun and graphic plot pressure vs. intruded volume is displayed on the screen. After the high pressure run is complete, the low- and high-pressure data files of the same sample are merged. The volume is then displayed and normalized for the weight of the sample and displayed as volume per weight. Since the weights of separate coating layers may be different for purposes of comparison, the results are normalized for weight.

### Pharmaceutically Active Agent

The tablet of the present invention includes at least one pharmaceutically active agent. What is meant by a “pharmaceutically active agent” is an agent (e.g., a compound) that is permitted or approved by the U.S. Food and Drug Administration, European Medicines Agency, or any successor entity thereof, for the oral treatment of a condition or disease. Suitable pharmaceutically active agents include, but are not limited to, analgesics, anti-inflammatory agents, antihistamines, antibiotics (e.g., antibacterial, antiviral, and antifungal agents), antidepressants, antidiabetic agents, antispasmodics, appetite suppressants, bronchodilators, cardiovascular treating agents (e.g., statins), central nervous system treating agents, cough suppressants, decongestants, diuretics, expectorants, gastrointestinal treating agents, anesthetics, mucolytics, muscle relaxants, osteoporosis treating agents, stimulants, nicotine, and sedatives.

Examples of suitable gastrointestinal treating agents include, but are not limited to: antacids such as aluminum-containing pharmaceutically active agents (e.g., aluminum carbonate, aluminum hydroxide, dihydroxyaluminum sodium carbonate, and aluminum phosphate), bicarbonate-containing pharmaceutically active agents, bismuth-containing pharmaceutically active agents (e.g., bismuth aluminate, bismuth carbonate, bismuth subcarbonate, bismuth subgallate, and bismuth subnitrate), calcium-containing pharmaceutically active agents (e.g., calcium carbonate), glycine, magnesium-containing pharmaceutically active agents (e.g., magaldrate, magnesium aluminosilicates, magnesium carbonate, magnesium glycinate, magnesium hydroxide, magnesium oxide, and magnesium trisilicate), phosphate-containing pharmaceutically active agents (e.g., aluminum phosphate and calcium phosphate), potassium-containing pharmaceutically active agents (e.g., potassium bicarbonate), sodium-containing pharmaceutically active agents (e.g., sodium bicarbonate), and silicates; laxatives such as stool softeners (e.g., docusate) and stimulant laxatives (e.g., bisacodyl); H2 receptor antagonists, such as famotidine, ranitidine, cimetadine, and nizatidine; proton pump inhibitors such as omeprazole and lansoprazole; gastrointestinal cytoprotectives, such as sucraflate and misoprostol; gastrointestinal prokinetics such as prucalopride; antibiotics for H. pylori, such as clarithromycin, amoxicillin, tetracycline, and metronidazole; antidiarrheals, such as bismuth subsalicylate, kaolin, diphenoxylate, and loperamide; glycopyrrolate; analgesics, such as mesalamine; antiemetics such as ondansetron, cyclizine, diphenyhydroamine, dimenhydrinate, meclizine, promethazine, and hydroxyzine; probiotic bacteria including but not limited to lactobacilli; lactase; racecadotril; and antiflatulents such as polydimethylsiloxanes (e.g., dimethicone and simethicone, including those disclosed in U.S. Pat. Nos. 4,906,478, 5,275,822, and 6,103,260); isomers thereof; and pharmaceutically acceptable salts and prodrugs (e.g., esters) thereof.

Examples of suitable analgesics, anti-inflammatories, and antipyretics include, but are not limited to, non-steroidal anti-inflammatory drugs (NSAIDs) such as propionic acid derivatives (e.g., ibuprofen, naproxen, ketoprofen, flurbiprofen, fenbufen, fenoprofen, indoprofen, fluprofen, pirprofen, carprofen, oxaprozin, pranoprofen, and suprofen) and COX inhibitors such as celecoxib; acetaminophen; acetyl salicylic acid; acetic acid derivatives such as indomethacin, diclofenac, sulindac, and tolmetin; fenamic acid derivatives such as mefanamic acid, meclofenamic acid, and flufenamic acid; biphenylcarbodylic acid derivatives such as diflunisal and flufenisal; and oxicams such as piroxicam, sudoxicam, isoxicam, and meloxicam; isomers thereof; and pharmaceutically acceptable salts and prodrugs thereof.

Examples of antihistamines and decongestants, include, but are not limited to, bromopheniramine, chlorcyclizine, dexbrompheniramine, bromhexane, phenindamine, pheniramine, pyrilamine, thonzylamine, pripolidine, ephedrine, phenylephrine, pseudoephedrine, phenylpropanolamine, chlorpheniramine, dextromethorphan, diphenhydramine, doxylamine, astemizole, terfenadine, fexofenadine, naphazoline, oxymetazoline, montelukast, propylhexadrine, triprolidine, clemastine, acrivastine, promethazine, oxomemazine, mequitazine, buclizine, bromhexine, ketotifen, terfenadine, ebastine, oxatamide, xylomeazoline, loratadine, desloratadine, and cetirizine; isomers thereof; and pharmaceutically acceptable salts and esters thereof.

Examples of cough suppressants and expectorants include, but are not limited to, diphenhydramine, dextromethorphan, noscapine, clophedianol, menthol, benzonatate, ethylmorphone, codeine, acetylcysteine, carbocisteine, ambroxol, belladona alkaloids, sobrenol, guaiacol, and guaifenesin; isomers thereof; and pharmaceutically acceptable salts and prodrugs thereof.

Examples of muscle relaxants include, but are not limited to, cyclobenzaprine and chlorzoxazone metaxalone, and orphenadrine, methocarbamol; isomers thereof; and pharmaceutically acceptable salts and prodrugs thereof.

Examples of stimulants include, but are not limited to, caffeine.

Examples of sedatives include, but are not limited to sleep aids such as antihistamines (e.g., diphenhydramine), eszopiclone, and zolpidem; isomers thereof; and pharmaceutically acceptable salts and prodrugs thereof.

Examples of appetite suppressants include, but are not limited to, phenylpropanolamine, phentermine, and diethylcathinone; isomers thereof; and pharmaceutically acceptable salts and prodrugs thereof.

Examples of anesthetics (e.g., for the treatment of sore throat) include, but are not limited to dyclonene, benzocaine, and pectin; isomers thereof; and pharmaceutically acceptable salts and prodrugs thereof.

Examples of suitable statins include but are not limited to atorvastin, rosuvastatin, fluvastatin, lovastatin, simvustatin, atorvastatin, and pravastatin; isomers thereof; and pharmaceutically acceptable salts and prodrugs thereof.

As discussed above, the pharmaceutically active agents of the present invention may also be present in the form of pharmaceutically acceptable salts, such as acidic/anionic or basic/cationic salts. Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate and triethiodide. Pharmaceutically acceptable basic/cationic salts include, and are not limited to aluminum, benzathine, calcium, chloroprocaine, choline, diethanolamine, ethylenediamine, lithium, magnesium, meglumine, potassium, procaine, sodium and zinc.

As discussed above, the pharmaceutically active agents of the present invention may also be present in the form of prodrugs of the pharmaceutically active agents. In general, such prodrugs will be functional derivatives of the pharmaceutically active agent, which are readily convertible in vivo into the required pharmaceutically active agent. Conventional procedures for the selection and preparation of suitable prodrug derivatives are described, for example, in “Design of Prodrugs”, ed. H. Bundgaard, Elsevier, 1985. In addition to salts, the invention provides the esters, amides, and other protected or derivatized forms of the described compounds.

Where the pharmaceutically active agents according to this invention have at least one chiral center, they may accordingly exist as enantiomers. Where the pharmaceutically active agents possess two or more chiral centers, they may additionally exist as diastereomers. It is to be understood that all such isomers and mixtures thereof are encompassed within the scope of the present invention. Furthermore, some of the crystalline forms for the pharmaceutically active agents may exist as polymorphs and as such are intended to be included in the present invention. In addition, some of the pharmaceutically active agents may form solvates with water (e.g., hydrates) or common organic solvents, and such solvates are also intended to be encompassed within the scope of this invention.

In one embodiment, the pharmaceutically active agent or agents are present in the tablet in a therapeutically effective amount, which is an amount that produces the desired therapeutic response upon oral administration and can be readily determined by one skilled in the art. In determining such amounts, the particular pharmaceutically active agent being administered, the bioavailability characteristics of the pharmaceutically active agent, the dose regime, the age and weight of the patient, and other factors must be considered, as known in the art.

The pharmaceutically active agent may be present in various forms. For example, the pharmaceutically active agent may be dispersed at the molecular level, e.g. melted, within the granule prior to coating, or may be in the form of particles, which in turn may be coated. A second pharmaceutically active agent may be present in the coated particle, or uncoated in the tablet matrix. If the pharmaceutically active agent is in form of particles, the particles prior to coating, granulation, or layering typically have an average particle size of from about 1 to about 1000 microns. In one embodiment, such particles are crystals prior to coating, layering of granulation having an average particle size of from about 1 to about 300 microns. In another embodiment, the particles have an average particle size of from about 50 to about 2000 microns, such as from about 50 to about 1000 microns, such as from about 100 to about 800 microns.

If the second pharmaceutically active agent, which is not coated with the modified release coating of the present invention, has an objectionable taste, the second pharmaceutically active agent may be coated with a taste masking coating, as known in the art. Examples of suitable taste masking coatings are described in U.S. Pat. No. 4,851,226, U.S. Pat. No. 5,075,114, and U.S. Pat. No. 5,489,436. Commercially available taste masked pharmaceutically active agents may also be employed. For example, acetaminophen particles, which are encapsulated with ethylcellulose or other polymers by a coaccervation process, may be used in the present invention. Coaccervation-encapsulated acetaminophen may be purchased commercially from Eurand America, Inc. (Vandalia, Ohio) or from Circa Inc. (Dayton, Ohio).

The pharmaceutically active agent may be present in pure crystal form or in a granulated form prior to the addition of the modified release coating. Granulation techniques may be used to improve the flow characteristics or particle size of the pharmaceutically active agents to make it more suitable for compression or subsequent coating. Suitable binders for making the granulation include but are not limited to starch, polyvinylpyrrolidone, polymethacrylates, hydroxypropylmethylcellulose, and hydroxypropylcellulose. The particles including pharmaceutically active agent(s) may be made by cogranulating the pharmaceutically active agent(s) with suitable substrate particles via any of the granulation methods known in the art. Examples of such granulation method include, but are not limited to, high sheer wet granulation and fluid bed granulation such as rotary fluid bed granulation, the details of which are disclosed in, “The Theory and Practice of Industrial Pharmacy, 3^{rd} edition”, Chapter 11, Lachman, Leon et al., 1986.

In one embodiment, one or more pharmaceutically active agents or a portion of the pharmaceutically active agents may be bound to an ion exchange resin prior to the addition of the osmotic coating.

In one embodiment, the pharmaceutically active agent is capable of dissolution upon contact with a fluid such as water, stomach acid, intestinal fluid or the like. In another embodiment, the dissolution characteristics of the pharmaceutically active agent are modified (e.g., controlled, sustained, extended, retarded, prolonged, or delayed) when analyzed using USP dissolution apparatus 1 (baskets) and USP apparatus 2 (paddles) at 50-150 rpm in the appropriate media including but not limited to water, 0.1N HCL, pH 5.8 phosphate buffer, and pH 7.2 phosphate buffer.

### Use of Tablet

In one embodiment, the present invention features a method of treating an ailment, the method including orally administering the above-described tablet wherein the tablet includes an amount of the pharmaceutically active agent effective to treat the ailment. Examples of such ailments include, but are not limited to, pain (such as headaches, migraines, sore throat, cramps, back aches and muscle aches), fever, inflammation, upper respiratory disorders (such as cough and congestion), infections (such as bacterial and viral infections), depression, diabetes, obesity, cardiovascular disorders (such as high cholesterol, triglycerides, and blood pressure), gastrointestinal disorders (such as nausea, diarrhea, irritable bowel syndrome and gas), sleep disorders, osteoporosis, and nicotine dependence.

In one embodiment, the first pharmaceutically active agent is the same as the second pharmaceutically active agent. In one embodiment, the first pharmaceutically active agent is different from the second pharmaceutically active agent.

In one embodiment, the method is for the treatment of an upper respiratory disorder, wherein the first pharmaceutically active agent is selected from the group consisting of decongestants and the second pharmaceutically active agent is selected from antihistamines.

In this embodiment, the “unit dose” is typically accompanied by dosing directions, which instruct the patient to take an amount of the pharmaceutically active agent that may be a multiple of the unit dose depending on, e.g., the age or weight of the patient. Typically the unit dose volume will contain an amount of pharmaceutically active agent that is therapeutically effective for the smallest patient. For example, suitable unit dose volumes may include one tablet.

In one embodiment, the osmotic tablet is adapted to release the first pharmaceutically active agent for a period of at least four hours upon ingestion, such as at least eight hours upon ingestion, such as at least twelve hours upon ingestion, such as at least twenty-four hours upon ingestion.

### EXAMPLES

Specific embodiments of the present invention are illustrated by way of the following examples. This invention is not confined to the specific limitations set forth in these examples.

### Example 1

### Preparation of Tablet Cores Containing a First Pharmaceutically Active Agent

The materials in Table 1 were mixed using a 2 quart PK-type V-blender blender. A 254 mg tablet core containing 180 mg of pseudoephedrine was then prepared by compressing the tablet on a rotary tablet press using standard concave tooling at ⅜ inches.

TABLE 1
Tablet Core Formulation

| Material | G/Batch | Weight % in Core |
|---|---|---|
| Pseudoephedrine HCl | 706.80 | 70.68 |
| Sodium Chloride | 98.2 | 9.82 |
| Hydroxypropyl methylcellulose¹ | 30.0 | 3.0 |
| Microcrystalline Cellulose | 100.0 | 10.0 |
| Povidone² | 60.0 | 6.0 |
| Magnesium Stearate | 5.0 | 0.5 |

¹Commercially available from Dow Corporation in Midland, Michigan, USA as HPMC 2208 (K15M) ™
²Commercially available from ISP Technologies Inc., Wayne, NJ as Plasdone K29-32 ™

### Example 2

### Preparation of Coated Tablets Containing Osmotic Coating for Laser Drilling

Part A: Preparation of Solvent Based Osmotic Coating Solution: Cellulose acetate (commercially available from Eastman Corporation, Kingsport, Tenn. as Cellulose Acetate 298-10™) and hydroxypropyl cellulose (commercially available from the Aqualon division of the Hercules Corporation, Wilmington, Del. as Klucel EF™) were slowly added to a 6133 g batch of solution containing 90 percent acetone (5189 g) and 10 percent water (577 g). The solution was prepared at a 6 percent solids level and contained 368 g of total polymer (276 g of Cellulose Acetate and 92 g of Hydroxypropyl cellulose) in order to coat a 4600 g batch of tablet cores at an 8 percent weight gain.

Part B: Preparation of Aqueous Based Osmotic Coating: A coating dispersion containing ethylcellulose (commercially available in a 30% solids aqueous dispersion as from FMC Biopolymer in Philadelphia, Pa. as Aquacoat™) and PVA/PEG copolymer (commercially available from BASF Corporation in Florham Park, N.J. as Kollicoat IR™) was prepared. The dispersion was prepared at a 14.8% solids level by adding 375 g of purified water and 80 g of total polymer (72 g of Ethylcellulose and 8 g of PVA/PEG Copolymer) for a total application of 11.8% coating level on a 800 g batch of tablet cores.

Part C: Application of the Solvent Based Osmotic Coating: A 4600 g batch of tablet cores from Example 1 was charged into a Glatt 5/9 fluid bed coating unit equipped with a Wurster insert commercially available from the Glatt Air Techniques Corporation in Ramsey, N.J. The batch was processed and coated at a rate of 35 g/min, a temperature of 37° C., and an atomization air pressure of 2 bars using the solution from Example 2, Part A. The tablets were then dried at 60° C. for 48 hours.

Part D: Application of the Aqueous Based Osmotic Coating: A 800 g batch of tablet cores from Example 1 was charged into a O′Hara coating pan unit commercially available from O′Hara Technologies in Ontario, Canada. The batch was processed and coated at a rate of 9 g/min, a temperature of 40° C., and an atomization air pressure of 12 psi. using the ethylcellulose based solution from Example 2, Part B. The tablets were dried at 60° C. for 48 hours.

### Example 3

### Laser Drilling of Tablets

Coated tablets from both Example 2, Part C and Example 2, Part D were laser drilled with 2 circular openings at a size of 0.44 mm each on both the top portion and the bottom portion of the tablets. A transverse-excited atmospheric (TEA) CO₂ laser was used to drill through the osmotic coatings. The laser having a wavelength of approximately 10,600 nanometers was used, a pulse duration of approximately 10 microseconds, and a power density of approximately 197.5 W/cm² was used to produce the desired openings.

### Example 4

### Compression of Immediate Release Coating Containing Both First and Second Pharmaceutically Active Agents or Placebo

Part A: Preparation of Immediate Release Coating 1 Containing Pseudoephedrine: A blend was prepared using the formulation outlined below in Table 2. The pseudoephedrine, Avicel pH 101, Croscarmellose sodium, and colloidal silicon dioxide were manually passed through a 40 mesh screen. The materials were blended in a suitable plastic bag end-over-end for 3 minutes to form a mixture. The magnesium stearate was manually passed through a 40 mesh screen and added to the mixture and blended end-over-end for 1 minute.

TABLE 2
Immediate Release Coating 1

| | | Batch Weight | |
|---|---|---|---|
| | | (g) for 500 g | Wt Percent |
| Material | Mg/Tablet | Batch | (%) of Layer |
| Pseudoephedrine HCl | 60.0 | 120 | 24 |
| Microcrystalline Cellulose | 182.5 | 365 | 73 |
| (Avicel pH 101)¹ | | | |
| Croscarmellose Sodium | 5.0 | 10.0 | 2.0 |
| (Ac-Di-Sol)² | | | |
| Colloidal Silicon Dioxide | 1.25 | 2.5 | 0.5 |
| (Cab-O-Sil)³ | | | |
| Magnesium Stearate | 1.25 | 2.5 | 0.5 |
| Total | 250 | 500 | 100 |

¹Commercially available from FMC Biopolymer in Philadelphia, PA as Avicel pH101 ™
²Commercially available from FMC Biopolymer in Philadelphia, PA as Ac-Di-Sol ™
³Commercially available from the Cabot Corporation in Boston, MA as Cab-O-Sil ™

Part B: Preparation of Immediate Release Coating 2 Containing Cetirizine: A blend was prepared using the formulation outlined below in Table 3. The Cetirizine, Avicel pH 101, Croscarmellose sodium, and colloidal silicon dioxide were manually passed through a 40 mesh screen. The materials were blended in a suitable plastic bag end-over-end for 3 minutes to form a mixture. The magnesium stearate was manually passed through a 40 mesh screen and added to the mixture and blended end-over-end for 1 minute.

TABLE 3
Immediate Release Coating 2 Containing Cetirizine

| | | Batch Weight | Wt Percent |
|---|---|---|---|
| | | (g) for 500 g | (%) of |
| Material | Mg/Tablet | Batch | Layer |
| Cetirizine Dihydrochloride | 10.0 | 33.35 | 6.67 |
| Microcrystalline Cellulose | 134.39 | 447.95 | 89.59 |
| (Avicel pH 101)¹ | | | |
| Croscarmellose Sodium | 3.0 | 10.0 | 2.0 |
| (Ac-Di-Sol)² | | | |
| Colloidal Silicon Dioxide | 1.11 | 3.7 | 0.74 |
| (Cab-O-Sil) | | | |
| Magnesium Stearate | 1.5 | 5.0 | 1.0 |
| Total | 150 | 500 | 100 |

Part C: Preparation of Placebo Immediate Release Coating 3: A blend was prepared using the formulation outlined below in Table 4. Avicel pH 101, Croscarmellose sodium, and colloidal silicon dioxide were manually passed through a 40 mesh screen. The materials were blended in a suitable plastic bag end-over-end for 3 minutes to form a mixture. The magnesium stearate was manually passed through a 40 mesh screen and added to the mixture and blended end-over-end for 1 minute.

TABLE 4
Immediate Release Coating 3 (Placebo Layer for analysis)

| | | Batch Weight | |
|---|---|---|---|
| | | (g) for a 100 g | Wt Percent |
| Material | Mg/Tablet | batch | (%) of Layer 1 |
| Microcrystalline | 139.89 | 93.26 | 93.26 |
| Cellulose (Avicel pH | | | |
| 101)¹ | | | |
| Croscarmellose Sodium | 7.5 | 5.00 | 5.00 |
| (Ac-Di-Sol)² | | | |
| Colloidal Silicon Dioxide | 1.11 | 0.74 | 0.74 |
| (Cab-O-Sil) | | | |
| Magnesium Stearate | 1.5 | 1.00 | 1.00 |
| Total | 150.0 | 100.0 | 100.0 |

Part D: Compression of Immediate Release Coatings 1 and 2: The Immediate Release Coating 1 (250 mg) and Immediate Release Coating 2 (150 mg) portions were individually weighed and applied to the coated, laser drilled tablets from Example 3. The order of compression was performed in the following steps using 31/64 inch tooling: First, Immediate Release Coating 1 was added to the die as the bottom layer of the tablet, and compressed at a force of 1 kiloNewtons. The coated tablet core from Example 3 was then added to the die on top of the first bottom layer, and then the Immediate Release Coating 2 was applied to the top portion of the tablet and compressed at a force of 15 kiloNewtons, wherein each side, excluding the core, has an average thickness of about 1.389 mm. This is calculated by subtracting the diameter of the core tablet (⅜ inches) from the diameter of the total tablet tooling ( 31/64 inches). The thickness per side is then divided by 2.

Part E: Compression of Immediate Release Coatings 1 and 3: The Immediate Release Coating 1 (250 mg) and Immediate Release Coating 3 (150 mg) at 150.0 mg per tablet were individually weighed and applied to the coated, laser drilled tablets from Example 3. The order of compression was performed in the following steps: First, Immediate Release Coating 1 was added to the die as the bottom layer of the tablet, and compressed at a force of 1 kiloNewtons. The coated tablet core from Example 3 was then added to the die on top of the first bottom layer, and then the Immediate Release Coating 3 was applied to the top portion of the tablet and compressed at a force of 15 kiloNewtons, wherein both sides have an average thickness of ⅜ inches.

### Example 5

### Preparation of Coated Tablets with Semipermeable Osmotic Coating and Subsequent Immediate Release Coating

Part A: Preparation of Solvent Based Osmotic Coating Solution: 63.48 g of Ethylcellulose (commercially available from Dow Corporation, Midland, Mich. as Ethocel™), 31.54 g of hydroxypropyl cellulose (commercially available from the Aqualon division of the Hercules Corporation in Wilmington, Del. as Klucel EF™), and 13.28 g of Triacetin are slowly added to a 1963 g batch of solution (solvent plus polymers) containing 1856 g of total solvent, including 90 percent acetone (1670 g) and 10 percent water (185.6 g). The solution is prepared at a 5.5 percent solids level and contained 108 g of total polymer at a ratio of 63.18:31.54:31.28 of Ethocel:HPC:Triactetin in order to coat a 1892 g batch of tablet cores at an 5.4 percent coating level.

Part B: Application of the Solvent Based Osmotic Coating for Tablets without a Laser Drilled Orifice: A 1892 g batch of tablet cores from Example 1 is charged into a Glatt 5/9 fluid bed coating unit equipped with a Wurster insert commercially available from the Glatt Air Techniques Corporation in Ramsey, N.J. The batch is processed and coated at a rate of 35 g/min with 1963 g of solution from Part A, a temperature of 37° C., and an atomization air pressure of 2 bars using the solution from Example 2, Part A. The tablets are then dried at 60° C. for 48 hours.

Part C: Compression Coating of Osmotic Coated tablets without a Laser-Drilled Orifice with Pseudoephedrine and Cetirizine

The Immediate Release Coating composition from Example 4, Part A containing pseudoephedrine (at 254 mg per tablet layer), and the Immediate Release Coating composition from Example 4, Part B containing cetirizine (at 150 mg per tablet layer) are individually weighed and applied to the coated, tablets from Example 5, Part B. The order of compression is performed in the following steps using 31/64 inch tooling: First, the Immediate Release Coating 1 is added to the die as the bottom layer of the tablet, and compressed at a force of 1 kiloNewtons. The coated tablet core from Example 5, Part B is then added to the die on top of the first bottom layer, and then the Immediate Release Coating 2 is applied to the top portion of the tablet and compressed at a force of 15 kiloNewtons, wherein each side, excluding the core, has an average thickness of about 1.389 mm.

Part D: Compression Coating of Osmotic Coated Tablets Without a Laser-Drilled Orifice with Pseudoephedrine and Placebo

The Immediate Release Coating composition from Example 4, Part A containing pseudoephedrine (added at 254 mg per tablet layer), and the Immediate Release Coating composition from Example 4, Part C containing no pharmaceutically active agents (added at 150 mg per tablet layer) are individually weighed and applied to the coated, tablets from Example 5, Part B. The order of compression is performed in the following steps: First, the Immediate Release Coating 1 is added to the die as the bottom layer of the tablet, and compressed at a force of 1 kiloNewtons. The coated tablet core from Example 5, Part B is then added to the die on top of the first bottom layer, and then the Immediate Release Coating 3 is applied to the top portion of the tablet and compressed at a force of 15 kiloNewtons, wherein each side, excluding the core, has an average thickness of about 1.389 mm.

### Example 6

### Preparation of Matrix Tablet with an Osmotic Coating

Part A: Blending and Compression: The materials in Table 5 were mixed using a 2 quart PK type V-blender. A 180 mg tablet core was then prepared by compressing the tablet on a rotary tablet press using standard concave tooling at ⅜ inches.

TABLE 5
Matrix Tablet Core Formulation

| | G/Batch for | |
|---|---|---|
| | a 1000 g | Weight % |
| Material | Batch | in Core |
| Pseudoephedrine HCl | 706.8 | 70.68 |
| Sodium Chloride USP | 98.2 | 9.82 |
| Hydroxypropyl methylcellulose¹ | 30.0 | 3.0 |
| Microcrystalline Cellulose | 100.0 | 10.0 |
| Povidone² | 60.0 | 6.0 |
| Magnesium Stearate | 5.0 | 0.5 |
| Total | 1000 | 100 |

¹Commercially available from Dow Corporation in Midland, Michigan, USA as HPMC 2208 (K100-M) ™
²Commercially available from ISP Technologies Inc., Wayne, NJ as Plasdone K29-32 ™

Part B: Application of Osmotic Coating: A 2000 g batch of tablet cores from Example 6, Part A is charged into a Glatt 5/9 fluid bed coating unit equipped with a Wurster insert commercially available from the Glatt Air Techniques Corporation in Ramsey, N.J. The batch is coated and processed using a coating solution prepared according to the procedure in Example 2, Part A and coated at a rate of 35 g/min, a temperature of 37° C., and an atomization air pressure of 2 bars using the solution from Example 2, Part A. The tablets are then dried at 60° C. for 48 hours.

Part C: Laser Drilling: Coated tablets from Example 6, Part B are laser drilled with 2 circular openings at a size of 0.44 mm each on both the top portion and the bottom portion of the tablets. A transverse-excited atmospheric (TEA) CO₂ laser is used to drill through the osmotic coatings.

Part D: Compression Coating of Osmotic Coated tablets with a delayed Matrix Core: The Immediate Release Coating composition 1 from Example 4, Part A containing pseudoephedrine (250 mg), and the Immediate Release Coating 2 composition from Example 4, Part B (150 mg) containing cetirizine are individually weighed and applied to the coated, tablets from Example 6, Part C. The order of compression is performed in the following steps: First, the Immediate Release Coating 1 is added to the die as the bottom layer of the tablet, and compressed at a force of 1 kiloNewtons. The coated tablet core from Example 6, Part C is then added to the die on top of the first bottom layer, and then the Immediate Release Coating 2 is applied to the top portion of the tablet and compressed at a force of 15 kiloNewtons, wherein each side, excluding the core, has an average thickness of about 1.389 mm.

Part E: Compression Coating of Osmotic Coated tablets with a delayed Matrix Core: The Immediate Release Coating 1 composition from Example 4, Part A containing pseudoephedrine (250 mg) and the Immediate Release Coating composition 2 from Example 4, Part C (150 mg) are individually weighed and applied to the coated, tablets from Example 6, Part C. The order of compression is performed in the following steps: First, the Immediate Release Coating 1 is added to the die as the bottom layer of the tablet, and compressed at a force of 1 kiloNewtons. The coated tablet core from Example 6, Part C is then added to the die on top of the first bottom layer, and then the Immediate Release Coating 3 is applied to the top portion of the tablet and compressed at a force of 15 kiloNewtons, wherein each side, excluding the core, has an average thickness of about 1.389 mm.

### Example 7

### Dissolution Data

Part A: DI Water Dissolution Media Analysis: The tablets produced in Example 4, Part E were placed into USP Type II apparatus (Paddles, 50 RPM) containing 900 mL of deionized water at 37° C. A Program VK8000 auto sampler was utilized to remove 10 mL from each vessel at 5 minutes, 30 minutes 1, 2, 3, 4, 8, 12, 18, and 24 hours and analyze the pulled samples for Pseudoephedrine by UV spectroscopy.

TABLE 4
Dissolution Data of Tablets

% Pseudoephedrine Dissolved
(Average of 6 vessels)

| | Aqueous Coated | Solvent Coated |
|---|---|---|
| Time point | tablets - Example 4 | tablets - Example 4 |
| 5 minutes | 22.9 | 21.8 |
| 30 minutes | 24.3 | 26.1 |
| 1 hour | 24.8 | 27.0 |
| 2 hours | 26.6 | 29.0 |
| 4 hours | 30.3 | 31.8 |
| 8 hours | 42.0 | 46.7 |
| 12 hours | 56.1 | 64.3 |
| 18 hours | 73.2 | 87.0 |
| 24 hours | 85.6 | 96.9 |

The data demonstrates that both the aqueous coated tablets and solvent coated tablets had (i) an initial release of the immediate release dose of pseudoephedrine in the first 5 minutes and (ii) an extended release dose from 30 minutes through 24 hours.

What is claimed is:

1. A method of manufacturing an osmotic tablet comprising:
(i) compressing a tablet core comprising a first pharmaceutically active agent and a hydrophilic polymer;
(ii) applying an osmotic coating to the outer surface of said tablet core to form a coated tablet, wherein said osmotic coating comprises at least one opening exposing said tablet core; and
(iii) compressing an immediate release coating onto the surface of said coated tablet, wherein said release coating comprises a second pharmaceutically active agent.

2. A method of claim 1, wherein said first pharmaceutically active agent is the same as said second pharmaceutically active agent.

3. A method of claim 1, wherein said first pharmaceutically active agent is different from said second pharmaceutically active agent.

4. A method of claim 1, wherein said first pharmaceutically active agent is selected from the group consisting of pseudoephedrine, phenylephrine, and dextromethorphan.

5. A method of claim 1, wherein said second pharmaceutically active agent is selected from the group consisting of cetirizine, loratadine, and fexofenadine.

6. A method of claim 4, wherein said second pharmaceutically active agent is selected from the group consisting of cetirizine, loratadine, and fexofenadine.

7. A method of claim 1, wherein said first pharmaceutically active agent is pseudoephedrine and said second pharmaceutically active agent in cetirizine.

8. A method of claim 1, wherein said osmotic tablet is adapted to release said first pharmaceutically active agent in a substantially zero order manner upon ingestion.

9. A method of claim 1, wherein said osmotic tablet is adapted to release said first pharmaceutically active agent for a period of at least twelve hours upon ingestion.

10. A method of claim 7, wherein said osmotic tablet is adapted to release said first pharmaceutically active agent for a period of at least twelve hours upon ingestion.

11. A method of claim 1, wherein said immediate release coating has an average thickness of at least 250 microns.

12. A method of claim 1, wherein the immediate release coating has an average porosity of at least of at least 0.02 cc/g and a pore diameter range of from about 0.2 and 3 microns.

13. A method of claim 1 wherein said osmotic coating comprises at least two openings.

14. A method of claim 3, wherein said release coating comprises both said first pharmaceutically active agent and said second pharmaceutically active agent.

15. A method of claim 10, wherein said release coating comprises both said first pharmaceutically active agent and said second pharmaceutically active agent.

16. A method of claim 7, wherein said release coating comprises a first portion and a second portion, wherein said first portion comprises said first pharmaceutically active agent and said second portion comprises said second pharmaceutically active agent and wherein said portions contact each other at a center axis of said tablet.

17. A method of claim 14, wherein said release coating comprises a first portion and a second portion, wherein said first portion comprises said first pharmaceutically active agent and said second portion comprises said second pharmaceutically active agent and wherein said portions contact each other at a center axis of said tablet.

18. A method of claim 16, wherein one of said portions comprises a third pharmaceutically active agent.

19. An osmotic tablet manufactured according to the method of claim 1.

20. A method of administering a first pharmaceutically active agent and a second pharmaceutically active agent, said method comprising ingesting an osmotic tablet of claim 19.
